(19) United States
(12) Patent Application Publication
(10) Pub. No.: US 2022/0135594 A1
(43) Pub. Date: May 5, 2022

(54) METHODS OF SYNTHESIS AND INTERMEDIATES

(71) Applicant: MEDIMMUNE LIMITED, Cambridge (GB)

(72) Inventors: Philip Wilson HOWARD, Cambridge (GB); Thais CAILLEAU, Cambridge (GB)

(21) Appl. No.: 17/433,226

(22) PCT Filed: Feb. 25, 2020
(86) PCT No.: PCT/EP2020/054837

§ 371 Date: Aug. 23, 2021

(30) Foreign Application Priority Data
Feb. 25, 2019 (GB) 1902495.9

(51) Int. Cl. C07D 519/00

(52) U.S. Cl. CPC C07D 519/00

(57) ABSTRACT

A method of synthesising a compound of formula (I) from a compound of formula (II) wherein R² is phenyl, substituted at either the meta- or para-position by the group (III) where Q is an amino acid residue (—C(═O)—X¹—NH—), a di-amino acid residue (—C(═O)—X¹—X²—NH—) or a tri-amino acid residue (—C(═O)—X¹—X²—X³—NH—); Prot^{N3} is an amino protecting group; R^{2pre} is phenyl, substituted at the same position as R² by —NH₂; comprising reacting a compound of formula (II) with a compound of formula (IV) in the presence of HATU in dichloromethane or chloroform or a mixture thereof.

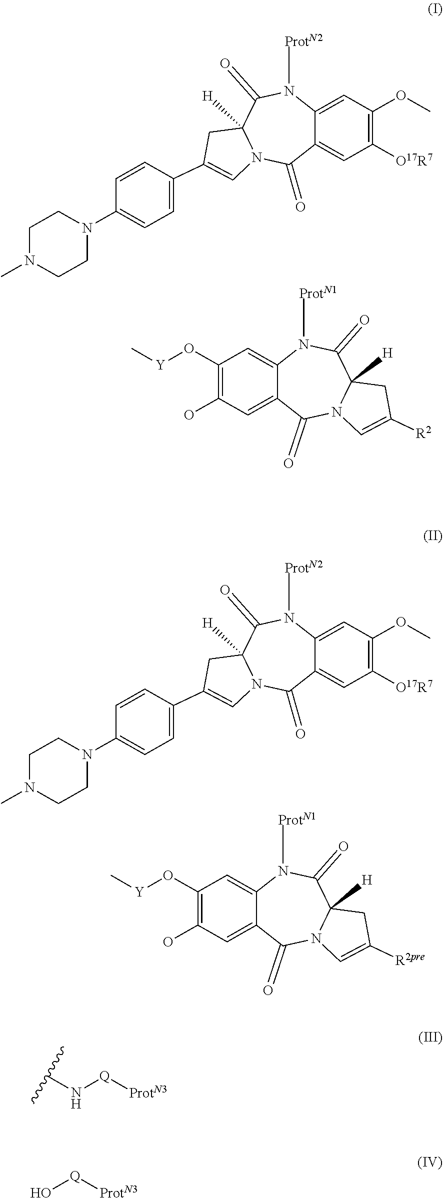

### METHODS OF SYNTHESIS AND INTERMEDIATES

The present invention relates to a key step in a method of synthesising dimeric PBD compounds bearing a linker for attachment to a cell binding agent.

### BACKGROUND

WO 2014/057073 discloses PBD dimers with a linker connected through the C2 position for the formation of PBD conjugates with cell binding agents, and in particular PBD antibody conjugates. Specifically, one of the compounds disclosed in WO 2014/057073 is compound B and its conjugates:

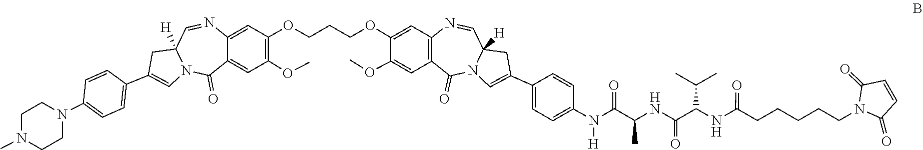

This compound is suitable for use in providing a PBD compound to a preferred site in a subject. A conjugate of this compound allows the release of an active PBD compound that does not retain any part of the linker.

### DISCLOSURE OF THE INVENTION

The present inventors have developed an efficient method of synthesising dimeric PBD compounds including a linking group as discussed above and bearing a methyl-piperazinyl-phenyl C2 substituent, which generally reduces or avoids racemisation, which method comprises coupling a protected peptide to an aniline PBD compound in the presence of HATU in dichloromethane or chloroform.

Accordingly, the present invention provides a method of synthesizing a compound of formula I:

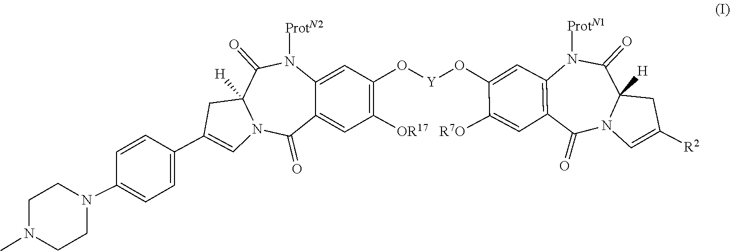

from a compound of formula II:

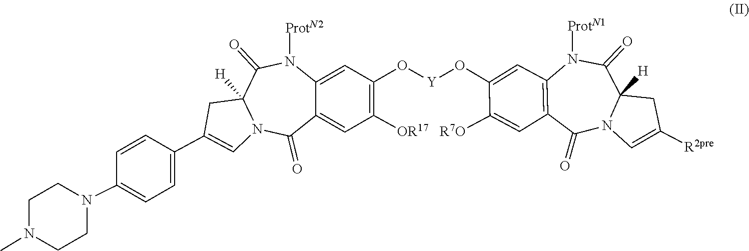

wherein

R² is phenyl, substituted at either the meta- or para-position by the group (III):

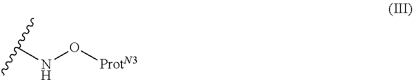

where Q is an amino acid residue (—C(═O)—X¹—NH—), a di-amino acid residue (—C(═O)—X¹—X²—NH—) or a tri-amino acid residue (—C(═O)—X¹—X²—X³—NH—);

Prot^{N3} is an amino protecting group;

R^{2pre} is phenyl, substituted at the same position as R² by —NH₂;

R⁷ is selected from C₁₋₄ alkyl and benzyl;

R¹⁷ is selected from C₁₋₄ alkyl and benzyl;

Y is a C₃₋₁₂ alkylene group, which chain may be interrupted by one or more heteroatoms, selected from O, S and NR^{N2} (where R^{N2} is H or C₁₋₄ alkyl), or an aromatic ring selected from benzene and pyridine;

Prot^{N1} and Prot^{N2} are independently selected from acetal nitrogen protecting groups; comprising reacting a compound of formula (II) with a compound of formula (IV):

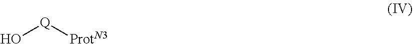

in the presence of HATU in dichloromethane or chloroform.

Epimerisation of the chiral centre of the activated amino acid residue during coupling reactions is a common problem. The racemisation of the chiral centre of the amino acid residue (—C(═O)—X¹—NH—) may occur to produce a diastereoisomeric mixture of products. This leads to the problem of additional steps needing to be carried out in order to isolate the desired isomer. This can be difficult or incur additional costs. It has been surprisingly found that coupling a protected peptide to an aniline PBD compound in the presence of HATU in dichloromethane or chloroform reduces or avoids racemisation, with a quick reaction time.

### Uses of Compounds of the Invention

Compounds made by the process of this invention are useful intermediates in the synthesis of PBD drug linkers, including some of those disclosed in WO 2014/057073.

### Definitions

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂— (propylene), —CH₂CH₂CH₂CH₂— (butylene), —CH₂CH₂CH₂CH₂CH₂— (pentylene) and —CH₂CH₂CH₂CH—₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C≡C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

Where the C₃₋₁₂ alkylene group is interrupted by a heteroatom, the subscript refers to the number of atoms in the chain including the heteroatoms. For example, the chain —C₂H₄—O—C₂H₄— would be a C5 group.

Where the C₃₋₁₂ alkylene group is interrupted by a heteroatom or an aromatic ring, the subscript refers to the number of atoms directly in the chain including the aromatic ring. For example, the chain

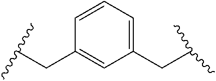 [SMILES: *Cc1cccc(C*)c1]

would be a C₅ group.

### Isomers

Certain compounds of the invention may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

### Amino Protecting Groups

Amino protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

Acetal nitrogen protecting groups are known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 884-887. Examples of acetal nitrogen protecting groups include: N-Hydroxymethyl, N-Methoxymethyl, N-Diethoxymethyl, N-(2-Chloroethoxy)methyl, N-[2-(Trimethylsilyl)ethoxy]methyl (SEM), N-t-Butoxymethyl, N-t-Butyldimethylsiloxymethyl, N-Pivaloyloxymethyl, N-Dimethylaminomethyl or N-2-Tetrahydropyranyl.

### Room Temperature

Room temperature as referenced in this application is between 18 and 25 degrees Celsius.

### Further Preferences

The following preferences apply to the invention as described above. The preferences may be combined together in any combination.

### Q

In one embodiment Q is an amino acid residue. The amino acid may a natural amino acids or a non-natural amino acid.

In one embodiment, Q is selected from: Phe, Lys, Val, Ala, Cit, Leu, lie, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:
^{NH}-Ala-Val-
^{NH}-Phe-Lys-,
^{NH}-Val-Ala-,
^{NH}-Val-Lys-,
^{NH}-Ala-Lys-,
^{NH}-Val-Cit-,
^{NH}-Phe-Cit-,
^{NH}-Leu-Cit-,
^{NH}-Ile-Cit-,
^{NH}-Phe-Arg-, and
^{NH}-Trp-Cit-;

where Cit is citrulline.

Preferably Q is selected from:
^{NH}-Ala-Val-
^{NH}-Phe-Lys-,
^{NH}-Val-Ala-,
^{NH}-Val-Lys-,
^{NH}-Ala-Lys-, and
^{NH}-Val-Cit-,

where Cit is citrulline.

Most preferably Q is ^{NH}-Ala-Val-.

Other dipeptide combinations of interest include:
^{NH}-Gly-Gly-,
^{NH} Pro-Pro-, and
^{NH}-Val-Glu-.

Other dipeptide combinations may be used, including those described by Dubowchik et al., Bioconjugate Chemistry, 2002, 13,855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:
^{NH}-Glu-Val-Ala-
^{NH}-Glu-Val-Cit-
^{NH}-αGlu-Val-Ala-
^{NH}-αGlu-Val-Cit-

In one embodiment, the amino acid side chain is chemically protected, where appropriate. The side chain protecting group may be a group as discussed below. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective groups in Organic Synthesis, Greene and Wuts.

Possible side chain protecting groups are shown below for those amino acids having reactive side chain functionality:
Arg: Z, Mtr, Tos;
Asn: Trt, Xan;
Asp: Bzl, t-Bu;
Cys: Acm, Bzl, Bzl-OMe, Bzl-Me, Trt;
Glu: Bzl, t-Bu;
Gln: Trt, Xan;
His: Boc, Dnp, Tos, Trt;
Lys: Boc, Z—Cl, Fmoc, Z;
Ser: Bzl, TBDMS, TBDPS;
Thr: Bz;
Trp: Boc;
Tyr: Bzl, Z, Z—Br.

The indication ^{NH} used above indicates which end of the di/tripeptide is attached to the aniline amino group.

### Prot^{N3}

Prot^{N3} is selected from Fmoc (fluorenylmethyloxycarbonyl), Teoc (2-(trimethylsilyl)ethoxycarbonyl) and Boc (t-butoxycarbonyl). In some embodiments, Prot^{N3} is selected from Fmoc and Teoc.

In a particularly preferred embodiment, Prot^{N3} is Fmoc.

R⁷ and R¹⁷

In some embodiments R⁷ is methyl.

In some embodiments R¹⁷ is methyl.

In some embodiments both R⁷ and R¹⁷ are methyl.

### Y

In some embodiments Y is a C₃₋₇ alkylene group with no substituents.

In some embodiments Y is a C₃, C₅ or C₇ alkylene group with no substituents.

In a particularly preferred embodiment Y is a C₃ alkylene group with no substituents.

Prot^{N1} and Prot^{N2}

In some embodiments Prot^{N1} and Prot^{N2} are both SEM (2-(Trimethylsilyl)ethoxymethyl).

### Compound of Formula IV

In some embodiments the compound of formula IV is FMoc-Val-Ala-OH.

### HATU

The reaction is carried out in the presence of HATU (1-[Bis(dimethylamino)methylene]-1H-1,2,3-triazolo[4,5-b]pyridinium 3-oxide hexafluorophosphate, also known as Hexafluorophosphate Azabenzotriazole Tetramethyl Uronium). In some embodiments the reaction is carried out with an equimolar amounts of HATU and compounds of formula II and IV.

HATU has the following structure:

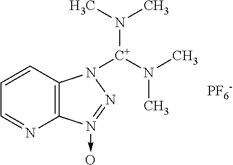

### HOBt

In some embodiments the reaction is carried out additionally in the presence of HOBt (Hydroxybenzotriazole). In some embodiments the molar amount of HOBt is the same as HATU.

In some embodiments the reaction is carried out with an equimolar amounts of HATU/HOBt and compounds of formula II and IV.

In a particularly preferred embodiment:
Q is -Ala-Val-;
Prot^{N3} is Fmoc;
R⁷ is methyl;
R¹⁷ is methyl;
Y is a C₃ alkylene; and
Prot^{N1} and Prot^{N2} are SEM.

### Solvent

The solvent for the reaction to be carried out in can be dichloromethane or chloroform.

In some embodiments, the solvent used is at least 75% dichloromethane, chloroform or a mixture thereof. In other embodiments, the solvent used is at least 80%, 85%, 90%, 95%, 99%, 99.5% or 99.9% dichloromethane, chloroform or a mixture thereof.

In some embodiments the only solvent is dichloromethane, chloroform or a mixture thereof.

In some embodiments the only solvent for the reaction is dichloromethane.

### General Experimental Methods

Optical rotations were measured on an ADP 220 polarimeter (Bellingham Stanley Ltd.) and concentrations (c) are given in g/100 mL. Melting points were measured using a digital melting point apparatus (Electrothermal). IR spectra were recorded on a Perkin-Elmer Spectrum 1000 FT IR Spectrometer. ¹H and ¹³C NMR spectra were acquired at 300 K using a Bruker Avance NMR spectrometer at 400 and 100 MHz, respectively. Chemical shifts are reported relative to TMS (δ=0.0 ppm), and signals are designated as s (singlet), d (doublet), t (triplet), dt (double triplet), dd (doublet of doublets), ddd (double doublet of doublets) or m (multiplet), with coupling constants given in Hertz (Hz). Thin Layer Chromatography (TLC) was performed on silica gel aluminium plates (Merck 60, F₂₅₄), and flash chromatography utilised silica gel (Merck 60, 230-400 mesh ASTM). Except for the HOBt (NovaBiochem) and solid-supported reagents (Argonaut), all other chemicals and solvents were purchased from Sigma-Aldrich and were used as supplied without further purification.

### General LC/MS Conditions:

The LC/MS_{(3 min)} conditions for the 3 minute run were as follows:

LCMS data were obtained using a Shimadzu Nexera series LC/MS with a Shimadzu LCMS-2020 quadrupole MS, with Electrospray ionisation. Mobile phase A—0.1% formic acid in water. Mobile phase B—0.1% formic acid in acetonitrile.

3 minute run gradient: Initial composition was 5% B held over 0.25 min, then increase from 5% B to 100% B over a 2 min period. The composition was held for 0.50 min at 100% B, then returned to 5% B in 0.05 minutes and hold there for 0.05 min. Total gradient run time equals 3 min. Flow rate 0.8 mL/min. Wavelength detection range: 190 to 800 nm. Oven temperature: 50° C. Column: Waters Acquity UPLC™ BEH Shield RP18 1.7 μm 2.1×50 mm at 50° C. fitted with Waters Acquity UPLC™ BEH Shield RP18 VanGuard Pre-column, 130 A, 1.7 μm, 2.1 mm×5 mm.

The LC/MS₍₆₀ₘᵢₙ₎ conditions for the 60 minute run were as follows:

LCMS data was obtained using a Thermo Scientific Dionex Ultimate 3000 Series liquid chromatography, RS pump, Autosampler, RS Diode array detector, RS Column oven, Q Exacutive mass spectrometer. Mobile phase A—0.1% formic acid in water. Mobile phase B—0.1% formic acid in acetonitrile.

Gradient: initial composition 20% B held over 1.25 min, then increase from 20% B to 60% B over a 53.75 min period. The composition was held for 2.5 min at 60% B, then returned to 20% B in 0.50 minutes and hold there for 2 min. Total gradient run time equals 60 min. Flow rate 0.5 mL/min. Wavelength detection range: 220 nm and 400 nm. Oven temperature: 50° C. Column: ACQUITY UPLC™ CSH C18, 1.7μ, 2.1×100 mm.

### Preparative HPLC:

HPLC (Shimadzu UFLC) was run using a mobile phase of water (0.1% formic acid) A and acetonitrile (0.1% formic acid) B. Wavelength detection range: 254 nm. Column: Phenomenex Gemini 5μ C18 150×21-20 mm. Gradient:
B
t=0 13%
t=15.00 95%
t=17.00 95%
t=17.10 13%
t=20.00 13%

Total gradient run time is 20 min; flow rate 20.00 mL/min.

### Synthesis of Key Intermediates

Compound 1 is compound 12 in WO 2014/057073 (see page 111).

### Synthesis of Compound 3

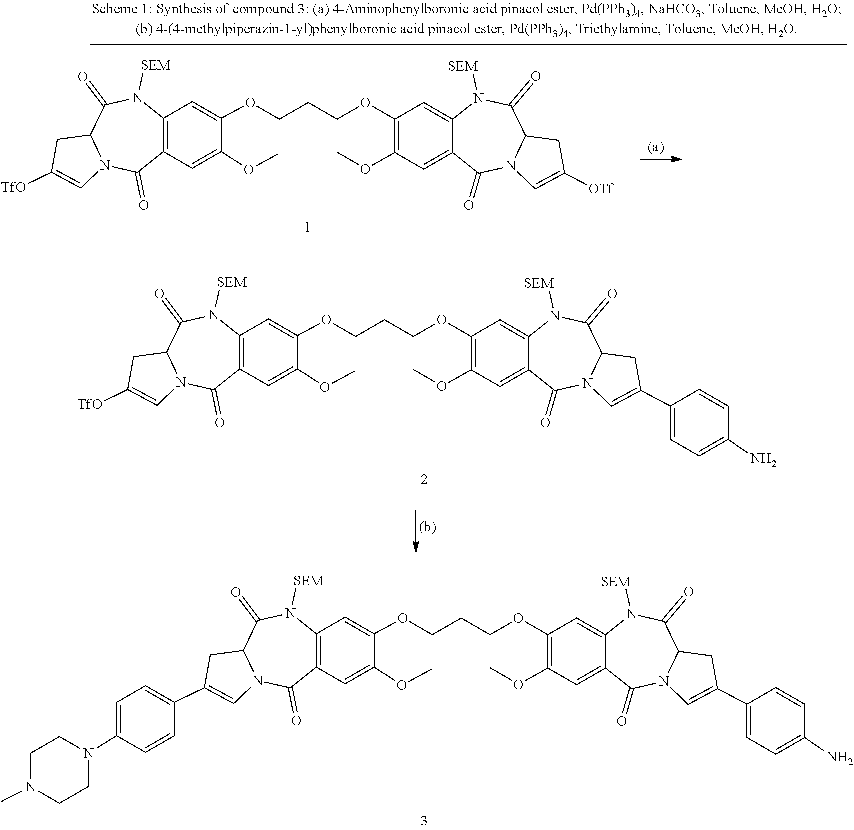 [SMILES: COc1cc2c(cc1OCCCOc1cc3c(cc1OC)C(=O)N1C=C(OS(=O)(=O)C(F)(F)F)CC1C(=O)N3COCC[Si](C)(C)C)N(COCC[Si](C)(C)C)C(=O)C1CC(OS(=O)(=O)C(F)(F)F)=CN1C2=O ; COc1cc2c(cc1OCCCOc1cc3c(cc1OC)C(=O)N1C=C(c4ccc(N)cc4)CC1C(=O)N3COCC[Si](C)(C)C)N(COCC[Si](C)(C)C)C(=O)C1CC(OS(=O)(=O)C(F)(F)F)=CN1C2=O ; COc1cc2c(cc1OCCCOc1cc3c(cc1OC)C(=O)N1C=C(c4ccc(N5CCN(C)CC5)cc4)CC1C(=O)N3COCC[Si](C)(C)C)N(COCC[Si](C)(C)C)C(=O)C1CC(c3ccc(N)cc3)=CN1C2=O]

### a) (S)-8-(3-(((S)-2-(4-aminophenyl)-7-methoxy-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-7-methoxy-5,11-dioxo-10-((2-(trimethylsilyl)ethoxy)methyl)-5,10,11,11a-tetrahydro-1H-benzo[e]pyrrolo[1,2-a][1,4]diazepin-2-yl trifluoromethanesulfonate (2)

Pd(PPh₃)₄ (310 mg, 0.26 mmol) was added to a stirred mixture of the bis-enol triflate 1 (15 g, 13.4 mmol), boronic ester (2.64 g, 12 mmol) and Na₂CO₃ (6.54 g, 61.7 mmol) in a 2:1:1 mixture of toluene/MeOH/H₂O (300 mL). The reaction mixture was allowed to stir at 30° C. under a nitrogen atmosphere for 16 h after which time all the boronic ester has consumed. The reaction mixture was then evaporated to dryness before the residue was taken up in CH₂Cl₂ (250 mL) and washed with H₂O (2×150 mL), brine (150 mL), dried (MgSO₄), filtered and evaporated under reduced pressure to provide the crude product. Purification by flash chromatography (gradient elution: 80:20 v/v Hexane/EtOAc to 60:40 v/v Hexane/EtOAc) afforded product 2 as a yellow foam (6.38 g, 45%). LC/MS_{(3 min)} 1.87 min (ES+) m/z=1060.35 [M+H]⁺.

### b) (S)-2-(4-aminophenyl)-7-methoxy-8-(3-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-10-((2-(trimethylsilyl)ethoxy)methyl)-1,11a-dihydro-5H-benzo[e]pyrrolo[1,2-a][1,4]diazepine-5,11(10H)-dione (3)

Pd(PPh₃)₄ (87 mg, 0.075 mmol) was added to a stirred mixture of the aniline-triflate 2 (4 g, 3.77 mmol), boronic ester (1.13 g, 3.77 mmol) and triethylamine (4.23 mL, 30.1 mmol) in a 2:1:1 mixture of toluene/MeOH/H₂O (10 mL). The reaction mixture was microwaved at 85° C. for 15 min. The resulting mixture was taken up in CH₂Cl₂ (75 mL) and washed with H₂O (2×50 mL), brine (50 mL), dried (MgSO₄), filtered and evaporated under reduced pressure to provide the crude product. Purification by flash chromatography (gradient elution: 80:20 v/v Hexane/EtOAc to 40:60 v/v Hexane/EtOAc) afforded product 3 as a yellow foam (3.107 g, 75%). LC/MS_{(3 min)} 1.39 min (ES+) m/z=1087.20 [M+H]⁺.

Peptide Coupling

### Synthesis of Compound 7

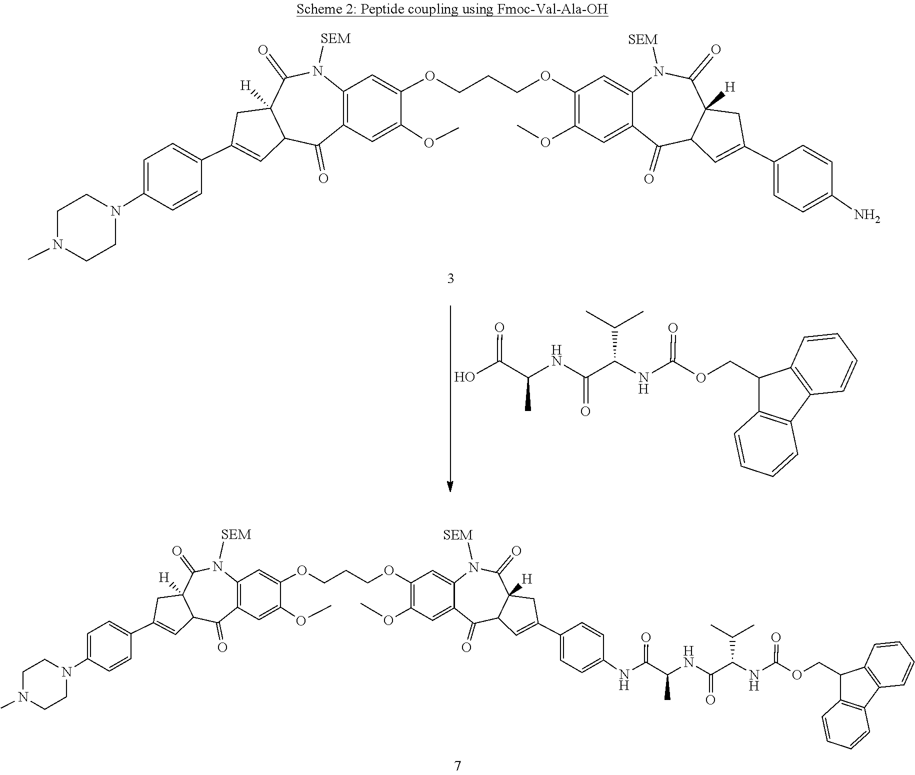 [SMILES: COc1cc2c(cc1OCCCOc1cc3c(cc1OC)C(=O)C1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(N)cc3)=CC1C2=O ; CC(C)[C@H](NC(=O)OCC1c2ccccc2-c2ccccc21)C(=O)N[C@@H](C)C(=O)O ; COc1cc2c(cc1OCCCOc1cc3c(cc1OC)C(=O)C1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(NC(=O)[C@H](C)NC(=O)[C@@H](NC(=O)OCC4c5ccccc5-c5ccccc54)C(C)C)cc3)=CC1C2=O]

### (9H-fluoren-9-yl)methyl ((S)-1-(((S)-1-((4-((S)-7-methoxy-8-(3-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-5,11-dioxo-10-((2-(trimethylsilyl)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (7)

To a solution of aniline 3 (500 mg, 0.459 mmol) in a solvent (20 mL) Fmoc-Val-Ala-OH (188 mg, 0.459 mmol) and a coupling agent (0.459 mmol) is added. The mixture is stirred at room temperature until completion. The reaction mixture is diluted with further solvent (50 ml), then washed with H₂O (2×50 ml), brine (50 ml), dried (MgSO₄) filtered and evaporated under reduced pressure to provide the crude product. Purification by flash chromatography (gradient elution: 100% CHCl₃ to 97/3 CHCl₃/MeOH) affords product 7 as a yellow foam.

Compound 7 was synthesised starting from SEM-dilactam 3 (Scheme 2). The Val-Ala dipeptide trigger was installed by coupling a protected dipeptide to the aniline with EEDQ.

The coupling of Fmoc-Val-Ala-OH with EEDQ was successful. However, upon LCMS analysis, the chromatogram displayed a second peak (13%) with a mass identical to that of the desired product. Epimerisation of the chiral centre on the activated amino acid during coupling reactions can be a problem and it is therefore hypothesised that the racemisation of the L-Alanine centre might have occurred to produce a diastereoisomeric mixture of products.

The table below shows multiple coupling conditions and the corresponding amounts of epimerised and desired product. SM corresponds to unreacted compound 3. These conditions were screened on the N-Me-piperazine intermediate 3 (Table 1).

TABLE 1
Conditions tried to achieve a peptide coupling. Amounts were
determined by LCMS analysis after a reaction time of 2.5 h.

| Coupling | | SM | Epimerised | Desired |
|---|---|---|---|---|
| agent | Solvent | (%) | product (%) | product (%) |
| EEDQ | CH₂Cl₂ | 0 | 14 | 86 |
| EEDQ | CH₂Cl₂/MeOH 100:1 | 41 | 16 | 43 |
| DCC | CH₂Cl₂ | 64 | 11 | 25 |
| DCC/DMAP | CH₂Cl₂ | 51 | 26 | 23 |
| DIC | CH₂Cl₂ | 56 | 17 | 27 |
| DIC/Oxyma | CH₂Cl₂ | 0 | 44 | 56 |
| HBTU/HOBt | CH₂Cl₂ | 57 | 6 | 37 |
| HBTU/HOBt | DMF | 20 | 40 | 40 |
| HATU/HOBt | DMF | 6 | 36 | 58 |

Nearly all attempted coupling conditions displayed various levels of epimerisation at the alanine chiral centre. Both reactions using DCC and DIC as coupling agents are very slow. Oxyma is as an efficient additive for peptide synthesis, known for its capacity to inhibit racemisation. However, despite speeding up the reaction rate, it produces almost equal amounts of epimers. Reactions employing HBTU/HOBt as coupling agents are both very slow. Interestingly, there is a significant difference in epimerisation levels observed depending on whether the reaction is run in CH₂Cl₂ or DMF.

The table below shows the coupling conditions using HATU and HATU/HOBt and the corresponding amounts of epimerised and desired product.

TABLE 2
Conditions tried to achieve a reduced racemising peptide coupling.
Amounts were determined by LCMS analysis after a reaction time of 2.5 h.

| Coupling | | SM | Epimerised | Desired |
|---|---|---|---|---|
| agent | Solvent | (%) | product (%) | product(%) |
| HATU | CH₂Cl₂ | 0 | 0 | 100 |
| HATU/HOBt | CH₂Cl₂ | 36 | 0 | 64 |

Equimolar amounts of HATU/Fmoc-Val-Ala-OH in CH₂Cl₂ proved to be the most efficient conditions, affording product 7 in 76% yield and as a single diastereomer by LCMS. Furthermore, there was no unreacted products in the reaction mixture (after a reaction time of 2.5 hours). The reaction using HATU/HOBt in DMF displayed high levels of racemisation (shown above in table 1), whereas similar conditions in CH₂Cl₂ afforded the product as a single enantiomer.

Therefore, it has been surprisingly found that the use of HATU as a coupling agent in dichloromethane or chloroform avoids racemisation, with a quicker reaction time.

### Synthesis of Compound 12

Subsequent reduction/deprotection of the SEM-dilactam 7 gives the corresponding imine 8. Fmoc deprotection using catalytic piperidine in DMF is completed within 10 minutes and after work up, product 9 is used in the next step without purification. Coupling of the acid spacer maleimide is achieved using EDCl.HCl and the final product is purified by reverse phase preparative HPLC to give pure compound 12.

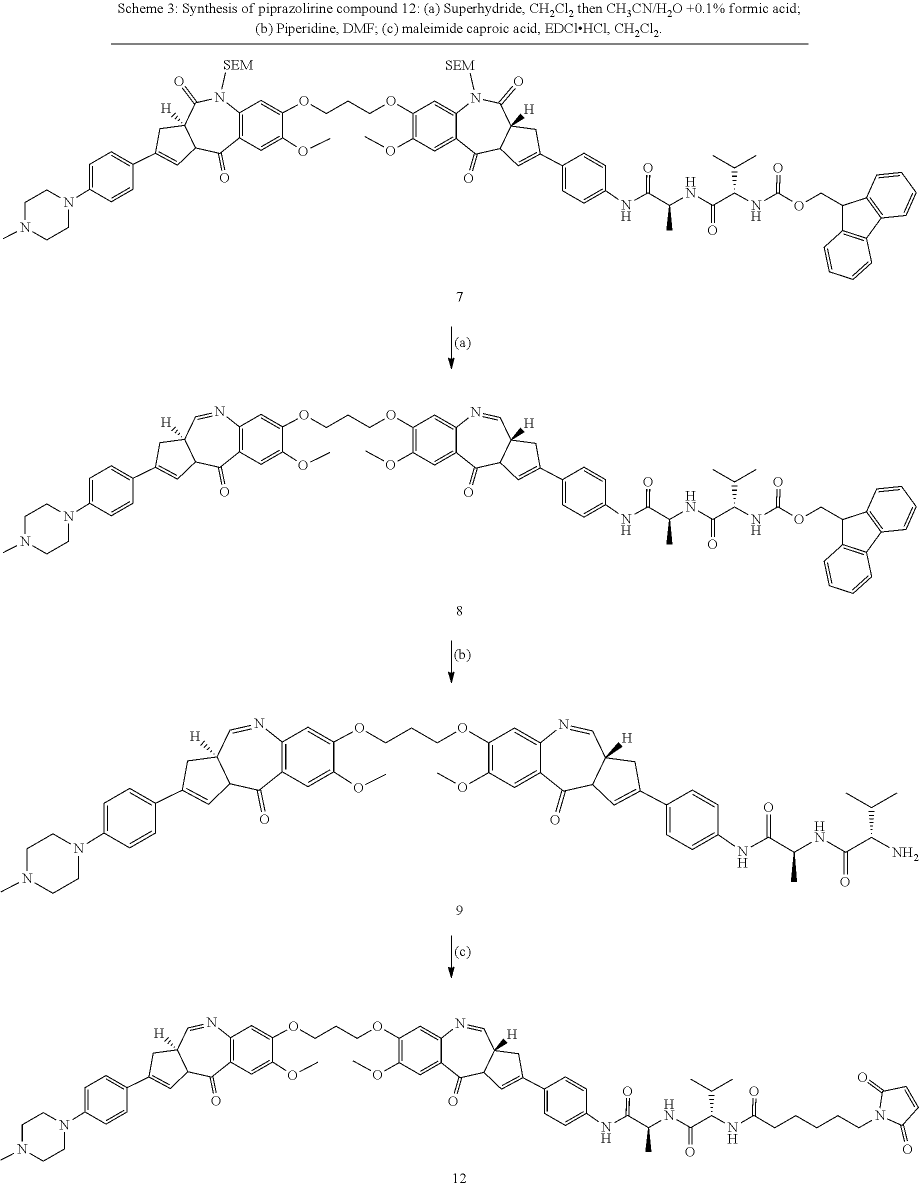

### Example 1

### Peptide Coupling Method

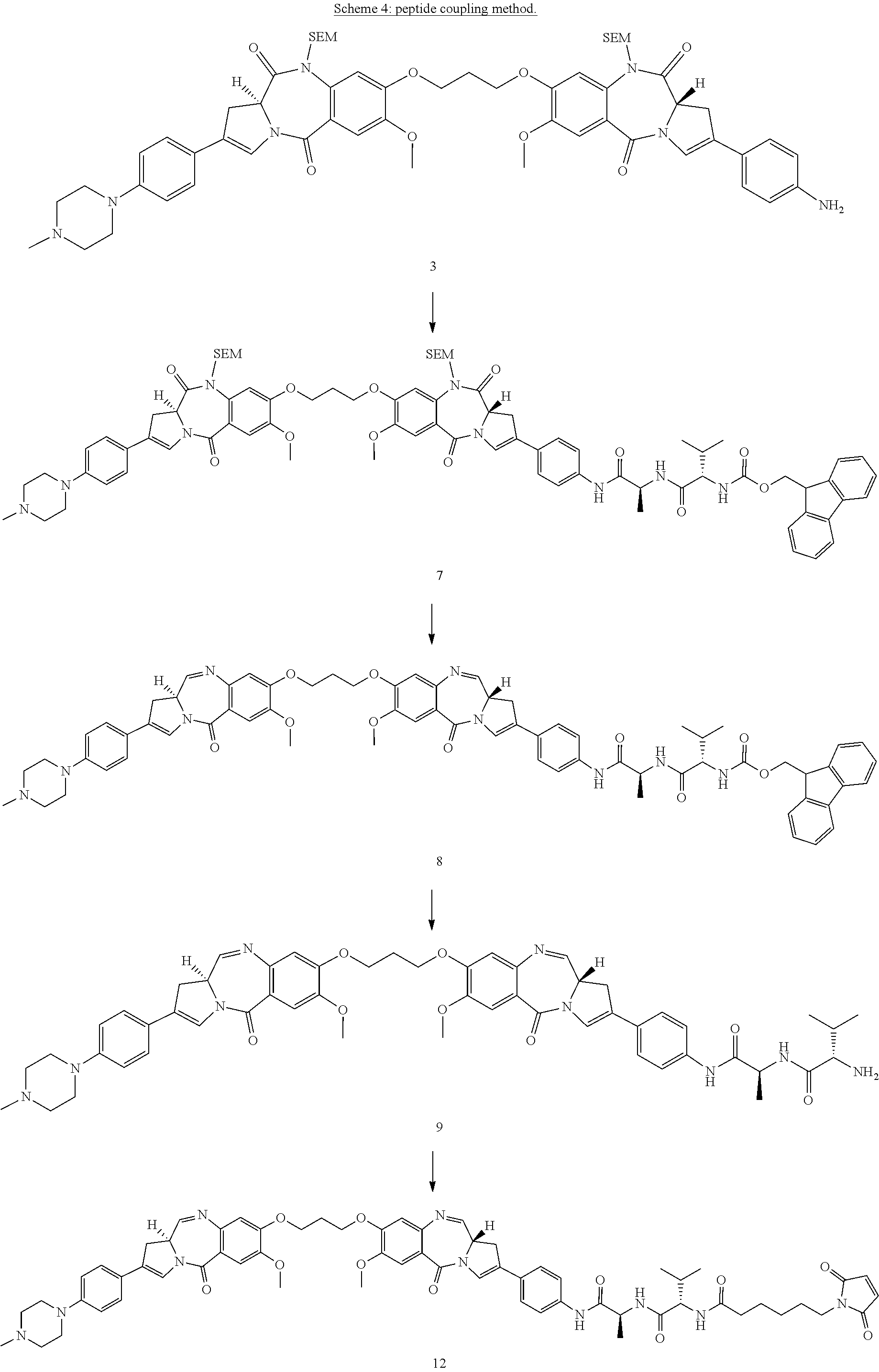

### a) Peptide Coupling

### (9H-fluoren-9-yl)methyl ((S)-1-(((S)-1-((4-((S)-7-methoxy-8-(3-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-5,11-dioxo-10-((2-(trimethylsilyl)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (7)

To a solution of aniline 3 (500 mg, 0.459 mmol) in dry CH₂Cl₂ (20 mL) was added Fmoc-Val-Ala-OH (188 mg, 0.459 mmol) and HATU (170 mg, 0.459 mmol). The mixture was stirred at room temperature until completion (≈1 h). The reaction mixture was diluted with CH₂Cl₂ (50 mL), then washed with H₂O (2×50 mL), brine (50 mL), dried (MgSO₄), filtered and evaporated under reduced pressure to provide the crude product. Purification by flash chromatography (gradient elution: 100% CHCl₃ to 97/3 CHCl₃/MeOH) afforded product 7 as a yellow foam (483 mg, 71% yield). LC/MS_{(60 min)} 41.47 min (ES+) m/z 1479.70 [M+H]⁺.

### b) Super-Hydride—Reduction/Deprotection

### (9H-fluoren-9-yl)methyl ((S)-1-(((S)-1-((4-((S)-7-methoxy-8-(3-(((S)-7-methoxy-2-(4-(4-methylpiperazin-1-yl)phenyl)-5-oxo-5,11a-dihydro-1H-pyrrolo[2,1-c][1,4]benzodiazepin-8-yl)oxy)propoxy)-5-oxo-5,11a-dihydro-1H-pyrrolo[2,1-c][1,4]benzodiazepin-2-yl)phenyl)amino)-1-oxopropan-2-yl)amino)-3-methyl-1-oxobutan-2-yl)carbamate (8)

A solution of Lithium triethylborohydride (Super-Hydride) (0.8 mL, 1M in THF) was added dropwise to a solution of SEM-dilactam 7 (483 mg, 0.327 mmol) in dry THF (5 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO₄, filtered and the solvent removed by rotary evaporation under reduced pressure. The crude product was dissolved in MeOH (18 mL), CH₂Cl₂ (9 mL), water (3 mL) and enough silica gel to form a thick stirring suspension. After 5 days, the suspension was filtered through a sintered funnel and washed with CH₂Cl₂/MeOH (9:1) (100 mL) until the elution of the product was complete. The organic layer was washed with brine (2×50 mL), dried with MgSO₄, filtered and the solvent removed by rotary evaporation under reduced pressure. Purification by silica gel column chromatography (isolera, CHCl₃/MeOH 98:2 to 80:20) afforded product 3 as a yellow solid (242 mg, 62.5% yield). LC/MS_{(60 min)} 20.72 min (ES+) m/z 1186.54 [M+H]⁺.

### c) Fmoc-Deprotection

### (S)-2-amino-N—((S)-1-((4-((S)-7-methoxy-8-(3-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)propoxy)-5-oxo-5,11a-dihydro-1H-benzo[e]pyrrolo[1,2-a][1,4]diazepin-2-yl)phenyl)amino)-1-oxopropan-2-yl)-3-methylbutanamide (9)

Excess piperidine was added (0.05 mL) to a solution of PBD 8 (242 mg, 0.2 mmol) in DMF (2 mL). The mixture was allowed to stir at room temperature for 20 min, at which point the reaction had gone to completion (as monitored by LC/MS). The reaction mixture was diluted with CH₂Cl₂ (50 mL) and the organic phase was washed with H₂O (2×50 mL) until complete piperidine removal. The organic phase was dried over MgSO₄, filtered and excess solvent removed by rotary evaporation under reduced pressure to afford crude product 9 which was used as such in the next step. LC/MS_{(3 min)} 1.03 min (ES+) m/z 483.00 [M+2H]²⁺.

### d) Amide Bond Formation

### 6-(2,5-dioxo-2,5-dihydro-1H-pyrrol-1-yl)-N—((S)-1-(((S)-1-((4-((S)-7-methoxy-8-(3-(((S)-7-methoxy-2-(4-(4-methylpiperazin-1-yl)phenyl)-5-oxo-5,11a-dihydro-1H-pyrrolo[2,1-c][1,4]benzodiazepin-8-yl)oxy)propoxy)-5-oxo-5,11a-dihydro-1H-pyrrolo[2,1-c][1,4]benzodiazepin-2-yl)phenyl)amino)-1-oxopropan-2-yl)amino)-3-methyl-1-oxobutan-2-yl)hexanamide (12)

EDCI hydrochloride (43 mg, 0.22 mmol) and 6-maleimidohexanoic acid (47 mg, 0.22 mmol) were added to a solution of 9 (crude) in dry CH₂Cl₂ (5 mL) under an argon atmosphere. Stirring was maintained until the reaction was complete (16 h). The reaction was diluted with CH₂Cl₂ (30 mL) and the organic phase was washed with H₂O (2×50 mL) and brine before being dried over MgSO₄, filtered and excess solvent removed by rotary evaporation under reduced pressure. The product was purified by careful silica gel chromatography chromatography (isolera, CHCl₃/MeOH 98:2 to 80:20) followed by reverse phase HPLC (water/CH₃CN) to remove shouldering impurities. Product 12 was isolated in 19.5% yield over two steps (46 mg). LC/MS_{(60 min)} 11.19 min (ES+) m/z (relative intensity) 1157.55 [M+H]⁺.

### Example 2

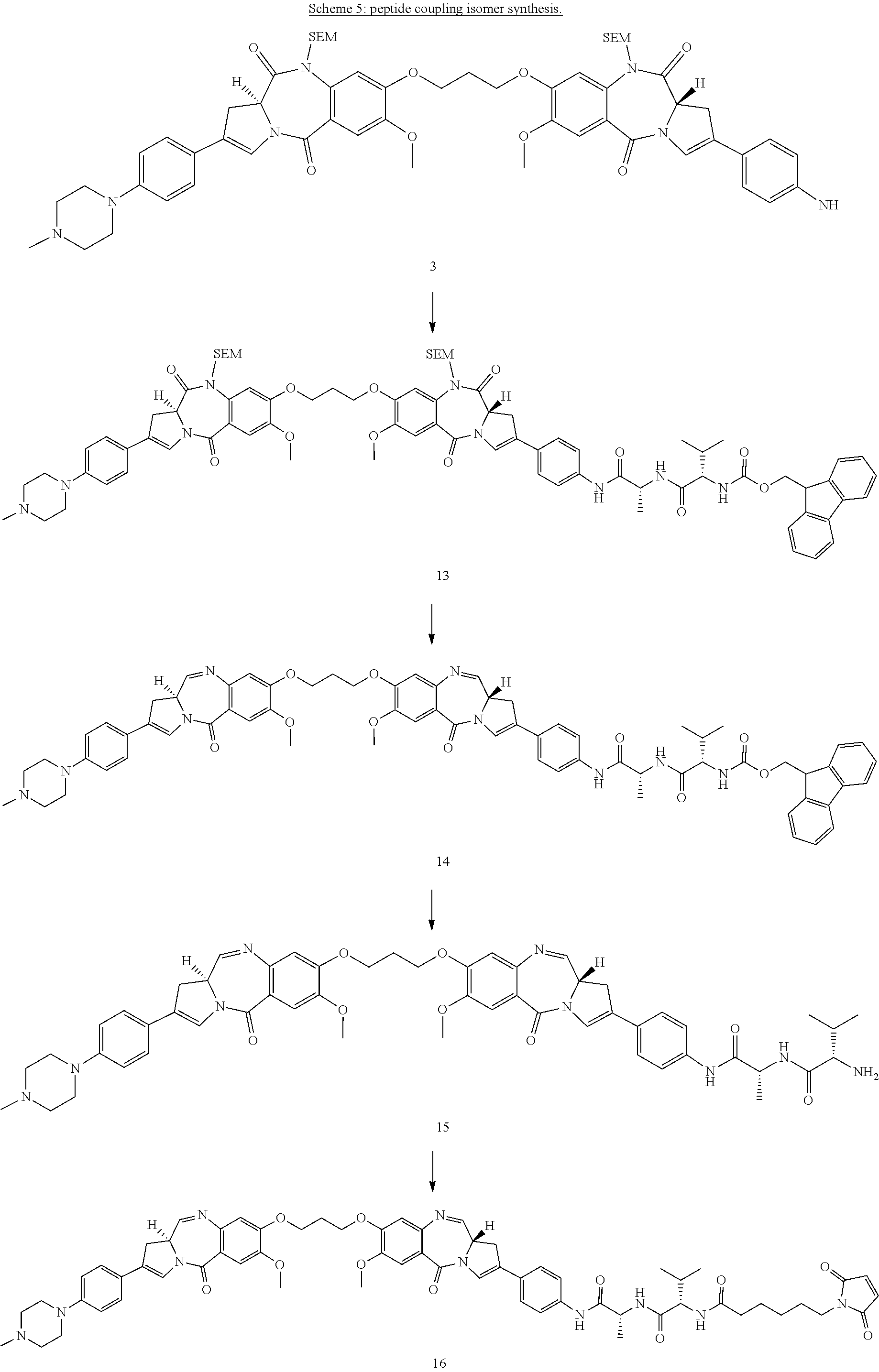

### a) Peptide Coupling

### (9H-fluoren-9-yl)methyl ((S)-1-(((R)-1-((4-((S)-7-methoxy-8-(3-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)propoxy)-5,11-dioxo-10-((2-(trimethylsilyl)ethoxy)methyl)-5,10,11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (13)

To a solution of aniline 3 (500 mg, 0.459 mmol) in dry CH₂Cl₂ (20 mL) was added Fmoc-Val-(D)-Ala-OH (188 mg, 0.459 mmol) and HATU (170 mg, 0.459 mmol). The mixture was stirred at room temperature until completion (≈1 h). The reaction mixture was diluted with CH₂Cl₂ (50 mL), then washed with H₂O (2×50 mL), brine (50 mL), dried (MgSO₄), filtered and evaporated under reduced pressure to provide the crude product. Purification by flash chromatography (gradient elution: 100% CHCl₃ to 97/3 CHCl₃/MeOH) afforded product 13 as a yellow foam (325 mg, 48% yield). LC/MS_{(60 min)} 40.77 min (ES+) m/z 1479.70 [M+H]⁺.

### b) Super-Hydride Reduction/Deprotection

### (9H-fluoren-9-yl)methyl ((S)-1-(((R)-1-((4-((S)-7-methoxy-8-(3-(((S)-7-methoxy-2-(4-(4-methylpiperazin-1-yl)phenyl)-5-oxo-5,11a-dihydro-1H-pyrrolo[2,1-c][1,4]benzodiazepin-8-yl)oxy)propoxy)-5-oxo-5,11a-dihydro-1H-pyrrolo[2,1-c][1,4]benzodiazepin-2-yl)phenyl)amino)-1-oxopropan-2-yl)amino)-3-methyl-1-oxobutan-2-yl)carbamate (14)

A solution of Lithium triethylborohydride (Super-Hydride) (0.55 mL, 1M in THF) was added dropwise to a solution of SEM-dilactam 13 (325 mg, 0.22 mmol) in dry THF (20 mL) at −78° C. under an argon atmosphere. The addition was completed over 5 minutes in order to maintain the internal temperature of the reaction mixture constant. After 40 minutes, an aliquot was quenched with water for LC/MS analysis, which revealed that the reaction was complete. Water (20 mL) was added to the reaction mixture and the cold bath was removed. The organic layer was extracted with CH₂Cl₂ (3×50 mL) and the combined organics were washed with brine (100 mL), dried with MgSO₄, filtered and the solvent removed by rotary evaporation under reduced pressure. The crude product was dissolved in MeOH (18 mL), CH₂Cl₂ (9 mL), water (3 mL) and enough silica gel to form a thick stirring suspension. After 5 days, the suspension was filtered through a sintered funnel and washed with CH₂Cl₂/MeOH (9:1) (100 mL) until the elution of the product was complete. The organic layer was washed with brine (2×50 mL), dried with MgSO₄, filtered and the solvent removed by rotary evaporation under reduced pressure. Purification by silica gel column chromatography (isolera, CHCl₃/MeOH 98:2 to 80:20) afforded product 14 as a yellow solid (216 mg, 83% yield). LC/MS_{(60 min)} 19.34 min (ES+) m/z 1186.70 [M+H]⁺.

### c) Fmoc-deprotection

### (S)-2-amino-N—((R)-1-((4-((S)-7-methoxy-8-(3-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)propoxy)-5-oxo-5,11a-dihydro-1H-benzo[e]pyrrolo[1,2-a][1,4]diazepin-2-yl)phenyl)amino)-1-oxopropan-2-yl)-3-methylbutanamide (15)

Excess piperidine was added (0.05 mL) to a solution of PBD 14 (215 mg, 0.18 mmol) in DMF (1.5 mL). The mixture was allowed to stir at room temperature for 20 min, at which point the reaction had gone to completion (as monitored by LC/MS). The reaction mixture was diluted with CH₂Cl₂ (50 mL) and the organic phase was washed with H₂O (2×50 mL) until complete piperidine removal. The organic phase was dried over MgSO₄, filtered and excess solvent removed by rotary evaporation under reduced pressure to afford crude product 15 which was used as such in the next step. LC/MS_{(3 min)} 1.02 min (ES+) m/z 483.00 [M+2H]²⁺.

### d) Amide Bond Formation

### 6-(2,5-dioxo-2,5-dihydro-1H-pyrrol-1-yl)-N—((S)-1-(((S)-1-((4-((S)-7-methoxy-8-(3-(((S)-7-methoxy-2-(4-(4-methylpiperazin-1-yl)phenyl)-5-oxo-5,11a-dihydro-1H-pyrrolo[2,1-c][1,4]benzodiazepin-8-yl)oxy)propoxy)-5-oxo-5,11a-dihydro-1H-pyrrolo[2,1-c][1,4]benzodiazepin-2-yl)phenyl)amino)-1-oxopropan-2-yl)amino)-3-methyl-1-oxobutan-2-yl)hexanamide (16)

EDCI hydrochloride (38 mg, 0.19 mmol) and 6-maleimidohexanoic acid (42 mg, 0.19 mmol) were added to a solution of 15 (crude) in dry CH₂Cl₂ (5 mL) under an argon atmosphere. Stirring was maintained until the reaction was complete (16 h). The reaction was diluted with CH₂Cl₂ (30 mL) and the organic phase was washed with H₂O (2×50 mL) and brine before being dried over MgSO₄, filtered and excess solvent removed by rotary evaporation under reduced pressure by rotary evaporation under reduced pressure. The product was purified by careful silica gel chromatography chromatography (isolera, CHCl₃/MeOH 98:2 to 80:20) followed by reverse phase HPLC to remove shouldering impurities. Product 16 was isolated in 26% yield over two steps (56 mg). LC/MS_{(60 min)} 10.87 min (ES+) m/z 1157.55 [M+H]⁺.

### Abbreviations

Ac acetyl
Acm acetamidomethyl
Boc di-tert-butyl dicarbonate
t-Bu tert-butyl
Bzl benzyl, where Bzl-OMe is methoxybenzyl and Bzl-Me is methylbenzene
DCC N,N′-Dicyclohexylcarbodiimide
DCM Dichloromethane
DIC N,N′-Diisopropylcarbodiimide
DMAP 4-Dimethylaminopyridine
DMF N,N-dimethylformamide
Dnp dinitrophenyl
EEDQ N-Ethoxycarbonyl-2-ethoxy-1,2-dihydroquinoline
EDCI N-(3-Dimethylaminopropyl)-N′-ethylcarbodiimide hydrochloride
Fmoc 9H-fluoren-9-ylmethoxycarbonyl
HBTU (2-(1H-benzotriazol-1-yl)-1,1,3,3-tetramethyluronium hexafluorophosphate
HOBt Hydroxybenzotriazole
HATU (1-[Bis(dimethylamino)methylene]-1H-1,2,3-triazolo[4,5-b]pyridinium 3-oxide hexafluorophosphate
Mtr 4-methoxy-2,3,6-trimethtylbenzenesulfonyl
Oxyma Ethyl cyanohydroxyiminoacetate
SEM (2-(Trimethylsilyl)ethoxymethyl).
TBDMS tert-butyldimethylsilyl
TBDPS tert-butyldiphenylsilyl
Teoc 2-(trimethylsilyl)ethoxycarbonyl
Tos tosyl
Trt trityl
Xan xanthyl

1. A method of synthesising a compound of formula I: 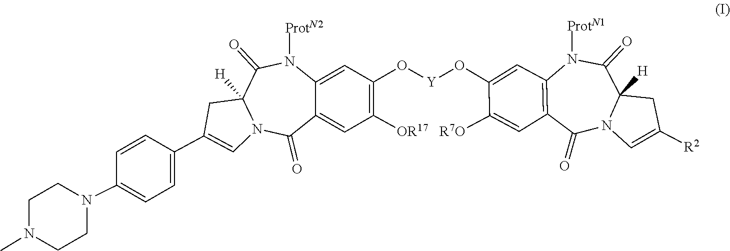
from a compound of formula II: 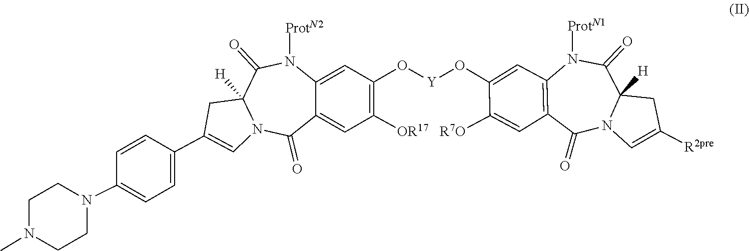
wherein
R² is phenyl, substituted at either the meta- or para-position by the group (III): 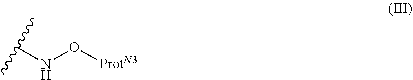
where Q is an amino acid residue (—C(═O)—X¹—NH—), a di-amino acid residue (—C(═O)—X¹—X²—NH—) or a tri-amino acid residue (—C(═O)—X¹—X²—X³—NH—);
Prot^{N3} is an amino protecting group;
R^{2pre} is phenyl, substituted at the same position as R² by —NH₂;
R⁷ is selected from C₁₋₄ alkyl and benzyl;
R¹⁷ is selected from C₁₋₄ alkyl and benzyl;
Y is a C₃₋₁₂ alkylene group, which chain may be interrupted by one or more heteroatoms, selected from O, S and NR^{N2} (where R^{N2} is H or C₁₋₄ alkyl), or an aromatic ring selected from benzene and pyridine;
Prot^{N1} and Prot^{N2} are independently selected from acetal nitrogen protecting groups;
comprising reacting a compound of formula (II) with a compound of formula (IV): 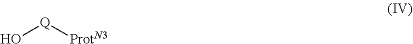
in the presence of HATU in dichloromethane or chloroform or a mixture thereof.

2. The method according to claim 1 wherein R² is phenyl, substituted at the para position by (III):

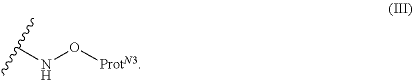

3. The method according to claim 1
wherein Q is selected from:
-Ala-Val-
-Phe-Lys-,
-Val-Ala-,
-Val-Lys-,
-Ala-Lys-,
-Val-Cit-,
-Phe-Cit-,
-Leu-Cit-,
-Ile-Cit-,
-Phe-Arg-, and
-Trp-Cit-;
where Cit is citrulline.

4.-5. (canceled)

6. The method according to claim 1, wherein Prot^{N3} is selected from Fmoc (fluorenylmethyloxycarbonyl), Teoc (2-(trimethylsilyl)ethoxycarbonyl) and Boc (t-butoxycarbonyl).

7. (canceled)

8. The method according to claim 1, wherein R⁷ is methyl, ethyl or propyl.

9. (canceled)

10. The method according to claim 1, wherein R⁷ is benzyl.

11. The method according to claim 1, wherein R¹⁷ is methyl, ethyl or propyl.

12. (canceled)

13. The method according to claim 1, wherein R¹⁷ is benzyl.

14. The method according to claim 1, wherein Y is a C₃₋₇ alkylene group with no substituents.

15. The method according to claim 14, wherein Y is a C₃, C₅ or C₇ alkylene group.

16. The method according to claim 15 wherein, Y is a C₃ alkylene group.

17. The method according to claim 1, wherein Prot^{N1} and Prot^{N2} are SEM (2-(Trimethylsilyl)ethoxymethyl).

18. The method according to claim 1, wherein the compound of formula IV is Fmoc-Val-Ala-OH.

19. The method according to claim 1, wherein the reaction is carried out at room temperature.

20. The method according to claim 1, wherein the reaction is carried out in the presence of Hydroxybenzotriazole.

21. The method according to claim 1, wherein:
Q is -Ala-Val-;
Prot^{N3} is Fmoc;
R⁷ is methyl;
R¹⁷ is methyl;
Y is a C₃ alkylene; and
Prot^{N1} and Prot^{N2} are SEM.

22. The method according to claim 1, wherein the compound of formula I is compound 7:

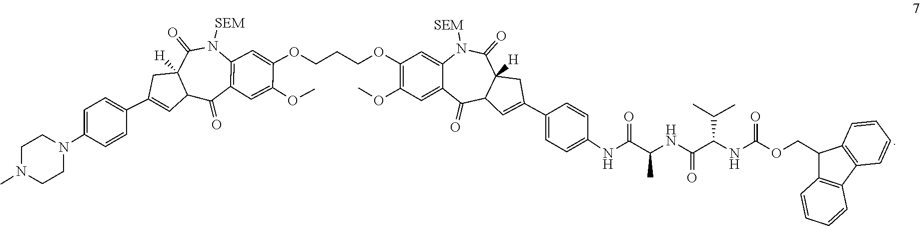 [SMILES: COc1cc2c(cc1OCCCOc1cc3c(cc1OC)C(=O)C1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(NC(=O)[C@H](C)NC(=O)[C@@H](NC(=O)OCC4c5ccccc5-c5ccccc54)C(C)C)cc3)=CC1C2=O]

23. The method according to claim 1, wherein the compound of formula I is compound 7: 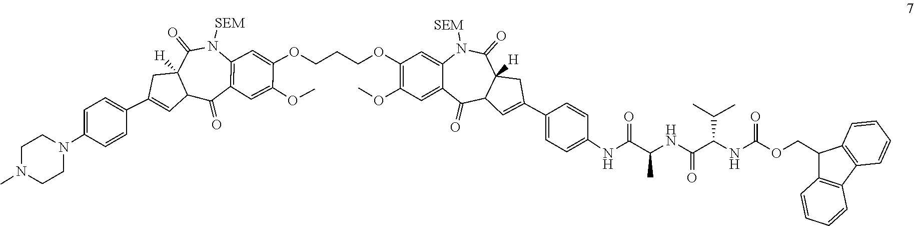 [SMILES: COc1cc2c(cc1OCCCOc1cc3c(cc1OC)C(=O)C1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(NC(=O)[C@H](C)NC(=O)[C@@H](NC(=O)OCC4c5ccccc5-c5ccccc54)C(C)C)cc3)=CC1C2=O]
and the compound of formula II is compound 3:

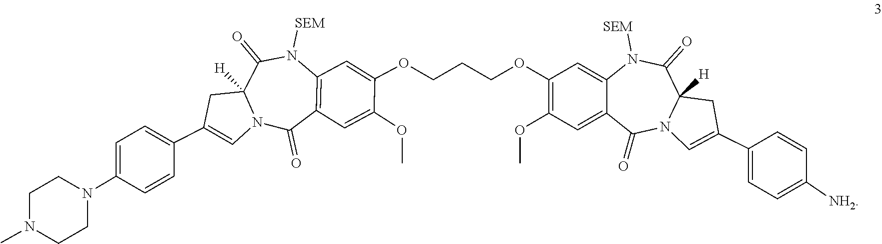 [SMILES: COc1cc2c(cc1OCCCOc1cc3c(cc1OC)C(=O)N1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(N)cc3)=CN1C2=O]

24. The method according to claim 1, wherein the reaction is carried out in dichloromethane.

25. The method according to claim 1, wherein the reaction is carried out in chloroform.
